SEQUENCE LISTING (3 sequences)

SEQ ID NO: 1 - protein, 9 aa, Artificial
  Synthetic
LLFGYPVYV

SEQ ID NO: 2 - protein, 9 aa, Artificial
  Synthetic
SLYNTVATL

SEQ ID NO: 3 - protein, 9 aa, Artificial
  Synthetic
NLVPMVATV

(19) United States
(12) Patent Application Publication
(10) Pub. No.: US 2007/0178532 A1
(43) Pub. Date: Aug. 2, 2007

(54) Identification, quantification, and characterization of t cells and t cell antigens

(75) Inventors: Steven Jacobson, Rockville, MD (US); Utano Takeuchi, Sapporo (JP); Yoshihisa Yamano, Kagoshima (JP)

Correspondence Address: LEYDIG, VOIT & MAYER, LTD., TWO PRUDENTIAL PLAZA, SUITE 4900, 180 NORTH STETSON AVENUE, CHICAGO, IL, 60601-6731, US

(73) Assignee: THE GOV.OF THE US AS REPRESENTED BY THE SECRETARY, Rockville, MD (US)

(21) Appl. No.: 10/551,126

(22) PCT Filed: Mar. 24, 2004
(86) PCT No.: PCT/US04/08960

§ 371 Date: Nov. 15, 2006

Related U.S. Application Data
(60) Provisional application No. 60/457,006, filed on Mar. 24, 2003.
(60) Provisional application No. 60/480,083, filed on Jun. 20, 2003.

(51) Int. Cl. G01N 33/567; C07H 21/04; C12P 21/06; C12N 5/08; C07K 14/435

(52) U.S. Cl. 435/7.2; 435/69.1; 435/372; 435/320.1; 530/350; 536/23.5

(57) ABSTRACT

Methods of quantifying and/or characterizing antigen-specific T cell populations, identifying T cell antigens and epitopes, and preparing targeted pharmaceutical compositions by (i) introducing a peptide into an antigen-presenting cell (APC) expressing a fusion protein comprising a major histocompatibility complex portion and a reporter peptide portion such that a complex forms between the fusion protein and the peptide and the peptide is displayed by the APC and (ii) contacting the APC with a population of cells, such that T cells in the population of cells that react with the peptide detectably internalize the complex; novel APCs useful in such methods; and related therapeutic and diagnostic methods.

### FIELD OF THE INVENTION

This invention pertains to methods of identifying and quantifying T cells; methods of identifying T cell antigens; methods of assessing the effects of such antigens on the immunological activity of T cells; compositions obtained by such methods; compositions used in performing such methods; and therapeutic applications of such methods and compositions.

### BACKGROUND OF THE INVENTION

Antigen-specific T cell interactions are important components of mammalian cellular immunity to microbial agents, self-proteins, and tumor antigens. A critical event in the initiation of a cellular immune response is the activation of T lymphocytes by T cell receptor (TCR) recognition of the peptide-major histocompatibility complex (MHC). This recognition initiates a precisely orchestrated cascade of molecular and cellular events that play an important part in the cellular immune response. In defining these interactions, the detection and quantitative analysis of epitope-specific T cell populations has been an important step toward understanding the cellular immune response in health and disease. Quantitative detection of T cell populations by tetramers has particularly proved useful for monitoring virus-specific T cell immunity in laboratory and clinical settings. However, an essential requirement for generating the tetramers is the need to identify a priori an immunodominant peptide known to bind the appropriate MHC.

Antigen-specific CD8+ T cells have been demonstrated to acquire peptide-MHC clusters through T cell receptor-mediated endocytosis upon specific antigen stimulation. Stinchcombe et al., Immunity, 15:751-761 (2001); Huang et al., Science, 286:952-954 (1999). However, apart from the mechanism of transfer itself, little is yet known about the biological consequences of antigen capture by T cells. Hudrisier et al., FASEB J., 16:477-486 (2002).

More significantly, until now there has been no description or suggestion of any practical applications arising from the discovery of this biological mechanism. Thus, for example, there has been before now no description of a method for quantifying antigen-specific T cell populations based upon T cell capture of detectable MHC-peptide clusters displayed by modified antigen presenting cells (APCs) or suggestion that such a method is possible. Similarly, there currently is no published description of a method for identifying antigenic peptides and epitopes by T cell internalization of such MHC-peptide clusters. Methods of characterizing T cell populations, methods of assessing disease, and methods of producing targeted therapeutic compositions by methods that capitalize on T cell capture of readily detectable peptide-MHC complexes similarly have not been reported, or even suggested, heretofore.

The invention described herein provides such new methods of quantifying and/or characterizing antigen-specific T cell populations, identifying T cell antigens and epitopes, and preparing targeted pharmaceutical compositions. These and other advantages of the invention, as well as additional inventive features, will be apparent from the description of the invention provided herein.

### BRIEF SUMMARY OF THE INVENTION

The invention provides an APC expressing at least two fusion proteins, wherein (i) each of the fusion proteins comprise an MHC molecule portion and a reporter peptide portion, and (ii) the reporter peptide portion of each fusion protein is detectably different from the reporter peptide portion of every other of the at least two fusion proteins and the MHC molecule portion of each fusion protein is different from the MHC molecule portion of every other of the at least two fusion proteins. Also provided by the invention is a human APC that expresses a fusion protein comprising a human leukocyte antigen (HLA)-A*201 portion and a reporter peptide portion.

The invention also provides a method for determining whether a T cell specific for a peptide is present in a population of cells. The method includes introducing into an APC, which expresses a fusion protein comprising (i) an MHC molecule portion and (ii) a reporter peptide portion, the peptide, such that a complex forms between the fusion protein and the peptide and the peptide is displayed by the APC. The APC displaying the complex is contacted with a population of cells, such that T cells in the population of cells specific for the antigen will detectably internalize the complex. Whether there are T cells in the population that are specific for the peptide is determined by determining whether T cells in the population of cells have detectably internalized the complex.

The invention further provides a method for quantifying the number of T cells, which are specific for an epitope of interest, in a population of cells comprising T cells. The method includes introducing a peptide comprising an epitope of interest into an antigen presenting cell (APC), which expresses a fusion protein comprising (i) a major histocompatibility complex (MHC) molecule portion which binds an epitope of interest and (ii) a reporter peptide portion, such that a complex forms between the fusion protein and the peptide and the peptide is displayed by the APC. The APC displaying the peptide is contacted with a population of cells comprising T cells, such that those T cells in the population of cells specific for the epitope detectably internalize the complex and T cells in the population specific for the epitope are quantified by enumerating the number of T cells in the population that have detectably internalized the complex.

The invention also provides a method for determining whether a peptide induces a T cell-mediated immune response. The peptide is introduced into an APC, which APC expresses a fusion protein comprising (i) an MHC molecule portion and (ii) a reporter peptide portion, under conditions where a complex can form between the fusion protein and the peptide and the peptide is displayed by the APC. The APC displaying the peptide is contacted with a population of T cells, such that T cells in the population specific for the peptide detectably internalize the complex. Whether the peptide induces a T cell-mediated immune response is determined by detecting whether cells in the population of T cells have internalized the complex.

The above method can be used to monitor the efficacy of treatment of a disease in a patient. The method comprises comparing the number of T cells, which are specific for one or more epitopes of interest, which can be from one or more antigens of interest, in a population of cells comprising T cells obtained from the patient before treatment and in a population of cells comprising T cells obtained from the patient during and/or after treatment. When the treatment induces a T cell-mediated response, an increase in the number of T cells after treatment as compared to the number of T cells before treatment indicates that the treatment is efficacious, whereas no change in the number of T cells or a decrease in the number of T cells after treatment as compared to the number of T cells before treatment indicates that the treatment is not efficacious. When the treatment inhibits a T cell-mediated response, no change in the number of T cells or a decrease in the number of T cells after treatment as compared to the number of T cells before treatment indicates that the treatment is efficacious, whereas an increase in the number of T cells after treatment as compared to the number of T cells before treatment indicates that the treatment is not efficacious.

The invention furthermore provides a method of evaluating the immunological effect of an antigen on the phenotypic or functional activity profile of a population of T cells. The method includes introducing into an APC, which expresses a fusion protein comprising (i) an MHC molecule portion and (ii) a reporter peptide portion, an antigen, such that a complex forms between the fusion protein and the antigen and the antigen is displayed by the APC and contacting the APC displaying the antigen with a population of T cells comprising T cells specific for the antigen and characterized by a phenotypic trait or functional trait, such that at least some of the T cells detectably internalize the complex. The population of T cells is thereupon characterized on the basis of (i) the phenotypic trait, (ii) the functional trait, (iii) a second phenotypic trait that differs from the phenotypic trait, (iv) a second functional trait that differs from the functional trait, or (v) any combination of (i)-(iv), whereupon the immunological effect of the antigen on the phenotypic or functional activity profile of the population of T cells is evaluated.

Additionally, the invention provides a method of preparing a targeted pharmaceutical composition for ameliorating a disease associated with T cell activity in a mammal. The method includes introducing a peptide into an APC expressing a MHC molecule portion-reporter peptide portion fusion protein, so that the peptide and fusion protein form a complex and the APC displays the peptide, and contacting the APC with a population of T cells, such that T cells in the population detectably internalize the complex. The peptide that induces a T cell-mediated immune response is characterized and an antigenic peptide comprising an amino acid sequence consisting essentially of the amino acid sequence of the amino acid sequence of the peptide is associated a molecule that inhibits the proliferation and/or activity of T cells, whereupon a targeted pharmaceutical composition for ameliorating a disease associated with T cell activity in a mammal is prepared.

### DETAILED DESCRIPTION OF THE INVENTION

The invention described herein provides, among other things, methods of quantifying and characterizing antigen-specific T cells, identifying T cell antigens and epitopes in larger peptides and populations of peptides, and methods of preparing pharmaceutical compositions that target T cells associated with particular diseases. The invention also provides novel compositions useful in the execution of such methods or that are obtained as a result of their application. Although the description of the invention provided herein separately discusses the several embodiments of the invention, it is to be understood that the description of any particular embodiment or feature of the invention can be applied to any other embodiment or feature, as appropriate, unless otherwise stated or clearly contradicted by context herein.

As mentioned above, the invention provides methods for quantifying the number of T cells specific for an antigen or epitope of interest in a population of cells. In such methods, a peptide that comprises an antigen or epitope of interest is introduced in an APC that expresses a fusion protein comprising an MHC molecule portion and a reporter peptide portion, such that a complex forms between the fusion protein and the peptide, which complex is displayed by the APC. The APC is contacted with a population of cells under conditions wherein T cells in the population specific for the antigen or epitope detectably internalize the complex. The number of T cells specific for the epitope or antigen is enumerated by determining the number of T cells in the cell population that have internalized the complex.

The above method can be used to monitor the efficacy of treatment of a disease in a patient. The method comprises comparing the number of T cells, which are specific for one or more epitopes of interest, which can be from one or more antigens of interest, in a population of cells comprising T cells obtained from the patient before treatment and in a population of cells comprising T cells obtained from the patient during and/or after treatment. When the treatment induces a T cell-mediated response, an increase in the number of T cells after treatment as compared to the number of T cells before treatment indicates that the treatment is efficacious, whereas no change in the number of T cells or a decrease in the number of T cells after treatment as compared to the number of T cells before treatment indicates that the treatment is not efficacious. When the treatment inhibits a T cell-mediated response, no change in the number of T cells or a decrease in the number of T cells after treatment as compared to the number of T cells before treatment indicates that the treatment is efficacious, whereas an increase in the number of T cells after treatment as compared to the number of T cells before treatment indicates that the treatment is not efficacious.

The disease can be mediated by a pathological T cell response. The disease can be an autoimmune disease, in which case the one or more epitopes of interest preferably is/are autoimmune epitope(s). Alternatively, the disease can be diabetes, in which case the one or more epitopes of interest preferably is/are epitopes from an islet of Langerhans cell. Still alternatively, the disease can be arthritis, in which case the one or more epitopes of interest is/are from collagen. When the disease is multiple sclerosis, the one or more epitopes of interest preferably is/are from myelin or an antigen of the central nervous system. When the disease results from infection with a virus, the one or more epitopes of interest preferably is/are from the virus, e.g., human immunodeficiency virus (HIV), a species of Vaccinia, hepatitis virus, or cytomegalovirus (CMV), whereas, when the disease results from infection with a bacterium, the one or more epitopes of interest is/are from the bacterium, e.g., a species of Chlamydia, Helicobacter or Mycobacteria, whereas, when the disease results from infection with a parasite, the one or more epitopes of interest is/are from the parasite. The treatment can comprise vaccination or immunization against the disease. Examples of diseases include anthrax, measles, rubella and cancer.

Any suitable T cell can be quantified by such methods of the invention. Thus, for example, the invention provides a method of quantifying the number of CD8+ T cells, such as the number of CD8+ cytotoxic T lymphocytes (CTLs) in the population of cells. The invention also provides a method of quantifying the number of CD4+ T helper (“Th”) cells in such a population, as well as a method of quantifying CD4+ CD8+ progenitor cells. The T cells also or alternatively can be characterized by any other suitable phenotypic characteristic (e.g., the presence of another cell marker, such as CD3, another cell marker protein discussed herein, or any other T cell marker known in the art) and/or the exhibition of any suitable functional characteristic (e.g., immunosuppressive activity). The population of cells can be of any suitable origin. Thus, for example, the population of cells can be obtained from a mammal, such as a primate, or, more particularly, a human. In other embodiments, the population of cells can comprise one or more types of synthetic cells (e.g., one or more recombinant cells) or a mixture of cells of natural and synthetic origins. In a particular embodiment, the cell population comprises or consists essentially of human peripheral blood mononuclear cells (PBMCs). The T cells enumerated by such methods can be considered “specific” for the epitope or antigen, although in some cases the T cells can be cross-reactive with other epitopes and/or antigens. Using the methods described above and elsewhere herein, the invention provides a method of identifying such cross-reactive T cells by performing such methods with multiple peptides until at least two peptides are identified that are detectably internalized by the T cells. Where two peptides that induce a cross-reactive T cell-mediated immune response are known, the invention provides a method for quantifying the number of cross-reactive T cells in a population of cells, applying the method of quantifying antigen/epitope-specific T cells in a population of cells, but repeating the method with the two peptides.

Any suitable peptide comprising a T cell antigen or epitope can be introduced into the APC for quantification by such methods. Typically and preferably, the peptide is a peptide of viral origin, a peptide of bacterial origin, an allergen, a peptide associated with a cancer, or a peptide associated with an autoimmune disease. The peptide can be introduced to the cells directly or as a component of a larger peptide, polypeptide, or protein that is processed by the APC (either due to the action of one or more of the APCs natural antigen-processing pathways or by some other action induced by the structure of the peptide and/or steps of the method—such as the inclusion of amino acid sequences that effect intein-like processing of the larger peptide, polypeptide, or protein and/or the co-administration of one or more proteolytic peptides that cleave the larger peptide, polypeptide, or protein to obtain the peptide). Typically, the amino acid sequence of the peptide is of a length consistent with the typical size of a mammalian T cell epitope (i.e., about 7-25 amino acids, usually about 8-15 amino acids, and often about 8-11 amino acids).

The peptide or larger peptide, polypeptide, or protein comprising the peptide can be introduced by any suitable method. For example, the APC can be pulsed with the peptide or a larger peptide, polypeptide, or protein comprising the peptide, in an amount sufficient for uptake and display of the peptide by the APC. Uptake of the peptide in the APC culture media can be facilitated by cell electroporation, treatment with cell permeabilizing agents, and/or other techniques known in the art for facilitating the uptale of peptides by such cells.

The peptide also or alternatively can be introduced to the APC by expressing a nucleic acid in the APC that encodes the peptide or a polypeptide that comprises the peptide. Such a nucleic acid typically is introduced into the APC as a part of a larger nucleic acid molecule and/or nucleic acid molecule-associated composition that facilitates the stable introduction and/or expression of the nucleic acid in the APC (i.e., a vector). Any suitable type of vector can be used for such delivery and expression of the nucleic acid. Thus, for example, the peptide can be introduced to the APC by expressing a nucleic acid in the APC that forms a part of a viral genome. The viral genome can be a wild-type viral genome, such that the peptide is a wild-type viral antigen. Alternatively, the peptide can be a nonviral polypeptide or a viral polypeptide not expressed by the wild-type counterpart of the viral genome, such that the viral genome can be characterized as a recombinant viral genome. Such virus and virus-derived nucleic acids can be delivered to the APC in recombinant viral particles or as nucleic acid vectors. Any suitable type of viral vector particle can be used to deliver the nucleic acids to the APC. Examples of suitable viral vector particles include human viral vectors, such as pox viral vectors (e.g., vaccinia viral vectors), adenoviral vectors, retroviral vectors (e.g., lentiviral vectors), adeno-associated viral vectors, herpes simplex viral (HSV) vectors, alphaviral vectors, and nonhuman viral vectors, such as recombinant avipox (e.g., fowlpox) vectors and avian influenza viral vectors. Alternatively, a nucleic acid vector (e.g., a plasmid, cosmid, phagemid, linear expression element, and the like) can be used to deliver the nucleic acid to the APC as a “naked” nucleic acid molecule or in association with a suitable transfection-facilitating agent (e.g., a liposome, one or more transfection-facilitating polypeptides, transfection-facilitating calcium phosphate compositions, and the like). Uptake of the viral vector or nucleic acid can be aided by techniques known in the art, such as electroporation, polyethylene glycol. (PEG)-facilitated transfection, biolistic (“gene gun”) delivery, injection, microinjection, transdermal delivery; etc.

The APC can be any suitable type of APC. Typically, the APC is a mammalian B lymphocyte, monocyte, macrophage, or dendritic cell. The APC also can be a T cell or modified T cell that presents MHC-antigen complexes to other T cells, although the APC preferably is not a T cell (B lymphocytes and dendritic cells typically are preferred). Preferably, the APC is a primate cell. Most preferably, the APC is a human cell. Typically and preferably, the methods of the invention are practiced with a population of such APCs of a size suitable for effecting transfer of the complex to a number of antigen-specific T cells in a desired period of time (e.g., about 30-120 minutes, about 45-90 minutes, or about 60 minutes), so that transfer of the peptide/complex can be readily detected in the T cells. For example, where the reporter peptide portion emits fluorescent radiation under known conditions, the method is preferably practiced with a number of APCs and a number of T cells sufficient to ensure rapid transfer of the fluorescent peptide complex to the T cells and detection therein by fluorescence microscopy or any other suitable fluorescence detection technique.

The APC is modified to express a fusion protein comprising an MHC molecule portion and a reporter peptide portion. Thus, the APC comprises a nucleic acid sequence that encodes the fusion protein. The APC can be obtained from a cell line wherein the fusion protein-encoding nucleic acid is stably integrated into the cellular genome or otherwise stably maintained in the APC. Alternatively, a nucleic acid encoding the fusion protein can be introduced into the APC by techniques described above with respect to the introduction of peptide-encoding nucleic acids (e.g., by transfection with a naked DNA vector, infection with a recombinant viral particle that expresses the fusion protein, or other suitable technique), prior to or in association with the introduction of the peptide or nucleic acid encoding the peptide.

The amino acid sequence of the portion of the fusion protein that forms the MHC molecule portion (1) corresponds to the amino acid sequence of any suitable wild-type vertebrate MHC molecule, (2) corresponds to a portion of such an MHC molecule that allows the fusion protein to form a complex with the antigen that is displayed by the APC, or (3) is an amino acid sequence that exhibits significant total amino acid sequence identity (e.g., at least about 85% amino acid sequence identity, at least about 90% amino acid sequence identity, at least about 95% identity, or more) with such a wild-type MHC molecule or MHC molecule portion, provided that the amino acid sequence allows the fusion protein to form a complex with the antigen that is displayed by the APC (methods for assessing amino acid sequence identity are described in, e.g., International Patent Application WO 02/083851 and U.S. Pat. No. 6,528,054). The MHC molecule portion can correspond to a class I MHC molecule, a class II MHC molecule, or a portion of either thereof that facilitates the formation of a complex with the antigen and the display the antigen by the APC. The MHC molecule portion preferably corresponds to a portion of a mammalian MHC molecule. More preferably, the MHC molecule portion corresponds to a primate MHC molecule. Most preferably, the MHC molecule portion corresponds to a human HLA class I or class II molecule. In a particularly preferred embodiment, the MHC molecule portion comprises the amino acid sequence of HLA*A201 human class I molecule. Typically, the fusion protein comprises a peptide portion that corresponds to one or more domains of an MHC chain. In such cases, the formation of the complex often requires the fusion protein to form a multimer (e.g., a heterodimer) with other MHC complex components expressed in the cell. For example, where the MHC molecule portion comprises a HLA*A201 amino acid sequence, the fusion protein desirably forms a heterodimer complex with beta2-microglobin in the endoplasmic reticulum of the human APC in which the fusion protein is expressed. Using techniques known in the art, the artisan can determine whether a fusion protein expressed in the APC forms such dimers and, if not, can make adjustments in the amino acid sequence of the fusion protein to promote the formation of a fusion protein-MHC molecule complex, and, in turn, the formation of a fusion protein-peptide complex that is displayed by the APC.

In addition to the MHC molecule portion, the fusion protein comprises a reporter peptide portion. The reporter peptide portion can comprise any amino acid sequence that allows the fusion protein to be readily detected. Thus, for example, the reporter peptide portion can comprise a functional portion of the β-galactosidase amino acid sequence, a functional portion of the chloramphenicol acetyltransferase (CAT) amino acid sequence, or a functional portion of a β-glucoronidase protein. In a preferred embodiment, the reporter peptide portion comprises an amino acid sequence of a fluorescent polypeptide, such as firefly luciferase, Aequorea victorta green fluorescent polypeptide, or a synthetic fluorescent polypeptide.

The fusion protein can include any other suitable number of additional domains, amino acids, and/or peptide portions other than the reporter peptide portion and the MHC molecule portion. For example, the fusion protein can include a linker sequence between the reporter peptide portion and the MHC molecule portion, such that the reporter peptide portion allows the complex to be detected when internalized by T cells, while also allowing the MHC molecule portion to associate with other MHC components and the peptide. Examples of suitable linker sequences are described in, e.g., International Patent Application WO 02/083851. The fusion protein also or alternative can include peptide portions that facilitate purification, targeting, cellular modification of the fusion protein, etc.

The APC are contacted with a population of cells under conditions wherein T cells in the population specific for the antigen or epitope detectably internalize the complex. As such, the population of test cells and the APC are mixed under conditions favorable for the formation of an APC-T cell “immunological synapse.” Thus, the cells are mixed in a media and cultured at a temperature suitable for supporting growth of the cells and formation of the immunological synapse for a suitable period of time to achieve a detectable transfer of the peptide from the APC to T cells specific for the peptide. Preferably, the cells are mixed for about 0.5-2 hours (e.g., about 0.5-1.5 hours, about 0.5-1 hour, or about 30 minutes). The method can comprise centrifuging the cells or applying other mixing techniques to bring the APC and target cells into contact with one another.

The number of T cells specific for the epitope or antigen in the population of cells is enumerated by determining the number of T cells in the cell population that have internalized the complex. The number of T cells that have internalized the complex can be determined by any suitable method that comprises detecting the reporter peptide portion of the fusion protein in the T cells. Thus, for example, the method can include subjecting the population of cells to fluorescence microscopy where the fusion protein comprises a GFP peptide portion to determine if any T cells in the population exhibit GFP-associated fluorescence. T cells that have internalized the complex can be further characterized by exhibiting one or more T cell markers (e.g., CD8, CD4, and/or CD3) in addition to a detectable characteristic associated with the reporter peptide portion. Flow cytometry analysis is particularly well-suited to such “dual characteristic” analysis.

Where the method is performed with a known antigen, the method can be characterized by comparing the number of T cells in a population of cells by enumerating the number of T cells acquiring the complex in the population or a sample of the population with the number of T cells obtained by applying one or more other standard assays to an identical population or second sample of the population. For example, the number of antigen-specific CTLs in a population of cells obtained by quantifying the CTLs in the population that capture a fusion protein-antigen complex from a modified APC displaying such a complex can be compared with the number of CTLs in an essentially identical population of cells determined by tetramer analysis. In another variation of the method, the number of CD4+ T cells in a population of cells is obtained by the inventive method and compared with the number of T cells in the population determined by cytokine (e.g., IFNγ) ELISPOT analysis. Preferably, the number of T cells obtained by the inventive T cell quantification method varies by less than about 25%, desirably less than about 20%, preferably less than about 15%, more preferably less than about 10%, and even more preferably less than about 5% from the number of T cells obtained by the standard quantification method (e.g., tetramer analysis).

The invention also provides methods for determining whether a peptide induces a T cell-mediated immune response. In such methods, a peptide is introduced into an APC that expresses a fusion protein comprising an MHC molecule portion and a reporter peptide portion, under conditions where a complex can form between the fusion protein and the peptide and the APC can display the complex. The APC is contacted with a population of T cells, such that T cells in the population specific for the peptide internalize the complex. Whether the peptide is a T cell antigen can then be determined by detecting whether T cells in the population internalize the complex.

The peptide in such methods can be a small peptide having a size typical of a T cell epitope (e.g., the peptide can be about 8-20 amino acids in length or a similar length such as those provide above). Alternatively, the peptide can be a portion of a larger peptide or polypeptide. Such a larger peptide or polypeptide can be a peptide or polypeptide known to induce a T cell response. Under such conditions, the method provides a process for identifying the antigenic portion of the polypeptide, such as a T cell epitope contained within such a polypeptide.

The peptide also can be a peptide or a portion of a polypeptide in a population (i.e., a pool, panel, group, or library) of peptides and/or polypeptides. Such a pool of peptides and/or polypeptides can collectively be known to induce a T cell immune response. For example, the peptide can comprise a sequence of amino acids that corresponds to a portion of a polypeptide that forms a portion of a viral capsid or viral envelope protein complex. Such a method preferably is performed to identify antigens in a population of peptides associated with a cancer, a virus, or an autoimmune disease. Such a method also can be used to identify T cell epitopes in a peptide known to be associated with a cancer, a virus, or an autoimmune disease.

Alternatively, the above-described method of assessing whether a peptide induces a T cell-mediated immune response can also be used to screen a pool of peptides and/or polypeptides that is previously uncharacterized with respect to the ability to induce a T cell immune response, to determine whether the pool comprises any peptides capable of inducing a T cell-mediated immune response. In such a method, the steps of (a) introducing a peptide into an APC expressing an MHC molecule portion/reporter peptide portion fusion protein that is capable of displaying a fusion protein-antigen complex, (b) contacting the APC with a population of T cells under conditions where the T cells can internalize a complex comprising a T cell antigen that the cells react to; and (c) determining whether T cells in the population have internalized the complex, are typically repeated until T cells in the population of T cells have internalized the complex. In each repetition of steps (a)-(c) the peptide introduced into the APC preferably has a unique amino acid sequence with respect to other peptides introduced into the APC in other repetitions of steps (a)-(c). Desirably, the peptides introduced into the APC in each repetition of steps (a)-(c) “overlap,” such that a portion of the amino acid sequence of each peptide is identical to a portion of the amino acid sequence of at least one other peptide introduced into the APC during the repetitions of steps (a)-(c). In such methods, the peptides can have any suitable amount of overlap. For example, the amount of identical amino acid sequence between overlapping peptides may be limited to only one amino acid residue or a few amino acid residues (e.g., about 1-5 amino acids). Alternatively, the amount of identical amino acids between overlapping peptides may form most or even essentially all of the peptides. For example, the method can include repeating step (a)-(c) with a panel of peptides where each peptide comprises a sequence identical to at least one other peptide in the panel of peptides except for one or two N-terminal and/or C-terminal amino acid residues that are added, deleted, or substituted with respect to the at least one other peptide. In a particular variation of the method, steps (a)-(c) are repeated with a panel of peptides of similar length that only differ with respect to the amino acid residue at a particular position in the sequence. Through such methods, the invention provides a technique for assessing the impact of the substitution of one or more amino acids on ability of a particular amino acid sequence to induce a T cell-mediated immune response. By applying such techniques, the invention provides a method for identifying a T cell antigen homolog of a wild-type T cell antigen and that differs from the wild-type by one or more amino acid substitutions. Synthetic T cell antigens identified by applying such a method can be associated with increased immunogenicity in a mammalian host to which they are administered or expressed in view of the lack of “self” amino acid sequences (e.g., in the case of cancer antigens and/or autoimmune disease-associated peptides) or viral amino acid sequences that evade host immune responses.

The invention also provides a method of preparing a targeted pharmaceutical composition for ameliorating a disease associated with T cell activity in a mammal. The method includes identifying a T cell antigen by T cell acquisition of an MHC molecule portion/reporter peptide portion-antigen complex from an APC displaying such a complex, characterizing the antigen portion of the complex, and preparing a targeted pharmaceutical composition comprising (i) an antigenic peptide that comprises an amino acid sequence substantially identical to the amino acid sequence of the antigen portion of the complex internalized in the T cells in intimate association with (ii) a substance toxic to such T cells.

Antigens identified by T cell acquisition of a fusion protein-antigen complex in accordance with elsewhere described embodiments of the invention can be characterized in any suitable manner that allows the antigen to be isolated or produced by peptide production techniques (e.g., isolation of the antigen from a cell comprising the antigen, expression of the antigen in a recombinant host cell, or chemical synthesis of the antigen). In most instances, particularly where the antigen is not isolated from a biological material, characterization of the antigen comprises determining the amino acid sequence of the antigen. Several suitable techniques for determining the amino acid sequence of a peptide are known in the art and can be employed for characterizing the antigen portion of such a complex.

An antigenic polypeptide comprising the amino acid sequence of the antigen or an amino acid sequence that is substantially identical to the antigen (e.g., at least about 85% identical, preferably at least about 90% identical, and more preferably at least about 95% identical to the antigen) can be prepared by any suitable technique including, e.g., expression of a nucleic acid sequence encoding the antigenic polypeptide in a suitable host cell and direct chemical synthesis. Preferably, the antigenic polypeptide comprises a sequence identical to most (e.g., at least about 80%, at least about 90%, or about 100%), if not all, of the amino acid sequence of the antigen portion of the complex. Where the antigenic portion comprises a sequence highly identical to, but different from, the antigen portion of the complex, the differences between the sequences are preferably associated with an enhancement of capture of the targeted pharmaceutical composition by the target T cells (e.g., by the introduction of one or more non-self amino acids into the amino acid sequence). Typically and preferably, the antigenic peptide comprises, consists essentially of, or consists entirely of, an amino acid sequence identical to the sequence of the antigen peptide) portion of the complex.

The antigenic polypeptide preferably is associated with a substance toxic to the T cells and/or that downregulates and/or suppresses the activity and/or proliferation of such T cells. Preferably, the substance is toxic to the T cells. The substance can be any suitable molecule, compound, or composition that induces such a biological effect. For example, the substance can be an organic molecule toxic to the T cells specific for the antigen, or that otherwise inhibits the proliferation and/or activity of such T cells. Examples of suitable molecules that can be associated with the antigenic peptide by conjugation or other association include corticosteroids such as prednisone, cytotoxic agents such as cyclophosphamide and methotrexate, and other chemotherapeutic agents such as azathioprine and cyclosporine. Alternatively, a fusion protein or polypeptide complex comprising a peptide portion or peptide comprising the amino acid sequence of the peptide in association with a second peptide that facilitates (enhances, promotes, and/or induces) apoptosis in the target T cells or fused to a second peptide portion that facilitates apoptosis in the target T cells. Thus, for example, the targeted pharmaceutical composition can comprise a fusion protein that comprises an antigenic peptide portion and a TNF-α peptide portion, IFNγ peptide portion, or a peptide portion that corresponds to the amino acid sequence of another apoptotic factor. The apoptotic factor peptide portion also can also correspond to a peptide that induces an apoptotic cascade in the target cells, such as a peptide that blocks NF-kB expression in the target cells. In other instances, the pharmaceutical composition can comprise a fusion protein comprising an antigenic peptide portion fused to a peptide that interferes with TNF-α and/or IL-1 signaling in the target cells. The antigenic peptide can form a part of any suitable fusion protein or any suitable peptide-toxic molecule composition, provided that the antigenic peptide facilitates the targeting of T cells specific for the antigen. The use of such targeted pharmaceutical compositions is particularly advantageous in the treatment of autoimmune diseases, where the inactivation and/or death of autoreactive T cells is/are desired.

As indicated above, immunomodulatory peptides comprising an amino acid sequence similar to the amino acid sequence of a T cell antigen identified by inventive methods of the invention can be prepared by varying the amino acid sequence of the antigen by one or more amino acid residues. Methods of producing such “altered peptide ligand” (APL) sequences are known in the art (see, e.g., Chen et al., J. Immunol., 157(9):3783-90 (1996), Kersh et al., J. Exp. Med. 184(4):1259-68 (1996), Bielekova et al., J. Mol. Med. 79(10):552-65 (2001), and Boisgerault et al., Methods Mol. Biol. 156:211-8 (2001)). Peptides comprising APL sequences can exhibit biological activity that differs from the T cell antigen (e.g., the APL peptide can act as an antagonist or partial agonist for activation of the T cells that internalize the antigen). Alternatively, an APL sequence can stimulate a T cell-mediated immune response similar to the response induced and/or promoted by an antigen identified by the methods of the invention. Thus, the invention provides a method of preparing a T cell antagonist or partial agonist by preparing an APL sequence that exhibits such activity through substitution, addition, or deletion of one or more amino acid-residues in the amino acid sequence of a T cell antigen identified by the inventive methods described elsewhere herein. In another method of the invention, a number of peptides including various APLs are (i) introduced into the APC (typically individually) and the APC mixed with the T cells or (ii) contacted directly with the T cells, to analyze the immunological activity of such APLs and/or identify immunomodulatory APL sequences. For example, an immunomodulatory APL sequence can be identified from an autoimmune disease-associated T cell antigen identified by T cell internalization of a fusion protein-antigen complex in accordance with the inventive methods described herein, by sequencing the antigen and modifying sequence by one or more substitutions, deletions, or additions to produce a panel of APLs. The panel of APLs are (i) introduced into the APC and the APC contacted with T cells specific for the antigen or (ii) allowed to directly contact the antigen-specific T cells, and the immunological impact of the APLs on the T cells is observed. These steps are repeated until an immunomodulatory APL sequence is identified. For example, the method can be repeated until one or more APLs are identified that act as antagonists for activation of the autoimmune disease-associated T cells.

The invention also provides new and useful methods for determining whether a T cell specific for an antigen is present in a population of cells. Similar to other inventive techniques described herein, such methods include introducing an antigen into an APC that expresses a fusion protein comprising an MHC molecule portion and a reporter peptide portion, such that a complex forms between the fusion protein and the antigen that is displayed by the APC, and contacting the APC displaying the complex with a population of cells, such that T cells in the population of cells specific for the antigen will detectably internalize the complex. Whether a T cell specific for the antigen is present in the population of cells can be determined by ascertaining whether T cells in the population of cells have internalized the complex. Such methods can be practiced with any suitable population of cells. Typically, the cells are obtained or derived from a vertebrate, and preferably the cells are derived or obtained from a mammal. Even more preferably, the cells are primate cells. Human cells, such as human PBMCs, are particularly preferred.

Such methods can be used to determine the presence of any suitable type of T cell. Thus, for example, the invention provides a method of determining whether a CD4+ antigen-specific T cell is present in the population of cells. The invention also, for example, provides a method of determining whether a CD8+, CD4+/CD8+, or CD3+ antigen-specific T cell is present in a population of T cells.

The invention further provides a method for identifying an antigen-specific T cell that includes repeating the steps of (a) introducing an antigen into an APC displaying an MHC molecule portion/reporter peptide portion fusion protein-antigen complex, (b) contacting such an APC with a population of cells that can include a T cell specific for the antigen, and (c) determining whether T cells in the population internalize the complex, until a T cell that internalizes the complex is identified. In each repetition of steps (a)-(c) in such methods, the population of cells that contacts the APC is different with respect to the population of cells that contacts the APC in all other repetitions of steps (a)-(c).

Additionally, the invention provides a method of evaluating the immunological effect of an antigen on the phenotypic profile of a population of T cells. The method includes introducing an antigen into APC that expresses a fusion protein comprising an MHC molecule portion and a reporter peptide portion, such that a complex forms between the fusion protein and the peptide that is displayed by the APCs and contacting the APCs with a population of T cells specific for the antigen and that are characterized by a phenotypic trait or a functional trait, such that at least some of the T cells internalize the complex. The immunological effect of the antigen on the T cells can be evaluated by characterizing the population of T cells on the basis of (i) the phenotypic trait, (ii) the functional trait, (iii) a second phenotypic trait that differs from the phenotypic trait, (iv) a second functional trait that differs from the functional trait, or (v) any combination of (i)-(iv).

Any suitable phenotypic characteristic associated with the T cells can be used to assess the immunological impact of the antigen on the phenotypic profile of the T cells. Typically, T cells are phenotypically characterized by one or more cell surface markers, such as CD27, CD28, CCR7, CD45RA, CD45RO, and combinations thereof. Thus, for example, the invention provides a method of assessing the immunological impact of an antigen on the phenotypic profile of a population of T cells on the basis CD27 and CD45RA expression.

The T cells also or alternatively can be characterized by any suitable functional characteristic associated with the T cells. For example, the method comprises characterizing the population of T cells with respect to perforin expression levels before, during, and/or after T cell internalization of the complex. Where the T cells are CTLs, the method can include characterizing the population of T cells on the basis of the cytolytic activity of the T cells with respect to one or more target cells (e.g., APCs pulsed with the antigen). Where the T cells are CD4+ T cells, the method can include characterizing the T cells on the basis of cytokine and/or chemokine production in the T cells. Thus, for example, the method can include characterizing the cells based on secretion of IFNγ, TNFα, and/or interleukins, such as IL-2 by the cells.

The phenotypic trait and/or functional trait can be a trait that detectably changes when the T cells induce and/or promote a therapeutic cellular immune response against disease-causing cells and the antigen is associated with the disease-causing cells. Under such conditions, the invention provides a method of assessing the effectiveness of a T cell-mediated treatment for the disease. For example, where the phenotypic and/or functional characteristics of the antigen-specific T cells retain or develop characteristics associated with T cells incapable of mounting an effective cell mediated immunological response after internalization of the complex, therapeutic methods that call for the use of a vaccine that activates such T cells is likely to be ineffective in the treatment of such a disease. The method can be repeated with various T cells and/or antigens, as necessary, to more fully evaluate the effectiveness of a T cell-mediated therapy against the disease. In such instances, the method can include evaluating the likelihood of success a T cell-mediated therapy and, in cases where such a therapy seems unlikely to succeed based on the phenotypic and/or functional characterization of the disease associated T cells by the inventive methods described herein, selecting a route of treatment that does not depend on the stimulation, promotion, and or increase in number of such T cells (e.g., application of surgical or radiation therapy techniques in the case of a cancer associated with such a T cell response to the antigen, or application of antiviral drugs, such as reverse transcriptase inhibitors, in the case of virus-associated disease associated with such a response). Moreover, the T cell phenotypic and/or functional trait evaluated in the method can be associated with the maturity level of T cells, such that the invention also provides a method for assessing the maturity level of a population of T cells. Such methods also are important in assessing the immune system of a mammal, such as a human patient.

In addition to the foregoing methods, the invention provides novel cells that can be used in the practice of such methods. Thus, for example, the invention provides a human APC that expresses a fusion protein comprising an MHC molecule portion and a reporter peptide portion that can display a complex of the fusion protein and the antigen that is recognized and internalized by T cells specific for the antigen when such T cells interact with the APC. More particularly, the invention provides a human APC that expresses a fusion protein comprising a human leukocyte antigen (HLA)-A*201 portion and a reporter peptide portion, which fusion protein is displayed by the APC and internalized by antigen-specific human T cells when a suitable antigen is introduced into the APC.

The invention additionally provides an APC that expresses at least two fusion proteins, wherein each of the fusion proteins comprise MHC molecule portion and a reporter peptide portion, wherein the reporter peptide portion of each fusion protein is detectably different from the reporter peptide portion of every other of the at least two fusion proteins and the MHC molecule portion of the each fusion protein is different from the MHC molecule portion of every other of the at least two fusion proteins. Such cells can be used to analyze a number of different peptides and cell populations in the methods of the invention described herein. The MHC molecule portions of the at least two fusion proteins can include MHC class I molecule amino acid sequences, MHC class II molecule amino acid sequences, or both. Thus, the MHC molecule portions of the at least two fusion proteins interact with peptides processed differently by the APC (e.g., endogenously versus exogenously) and/or can interact with antigens of significantly different characteristics. The reporter peptide portions of the at least two fusion proteins can be any suitable type of reporter peptide portions that exhibit detectably different characteristics when internalized by T cells. Preferably, the reporter peptide portions of the at least two fusion proteins are fluorescent proteins that fluoresce at detectably different wavelengths from one another.

The methods of the invention offer a number of advantages over currently used antigen-specific T cell screening methods (e.g., tetramers, ELISPOT, and other in vitro cytokine detection systems). Modified APCs that stably express suitable MHC molecule portion/reporter peptide portion fusion proteins can readily be prepared and maintained using widely available and relatively inexpensive techniques and peptides of interest can rapidly and easily be introduced into such cells (e.g., APCs can be pulsed with any peptide in a short 30 minutes incubation process) Moreover, the method is applicable to any suitable type of MHC allele and can be practiced with peptides that have not been characterized on the basis of their immune response (offering a particular advantage over tetramer analysis). Several additional applications arising from the inventive methods described herein will be readily apparent to those skilled in the art. For example, methods of analyzing the T cell-mediated immune response described herein can be used to analyze the suitability of a particular cell or tissue transfer with respect to the potential for rejection of the donor cells and/or tissue. Additionally, the invention provides novel methods of forming and studying the immunological synapse between an APC and T cells and methods for determining the kinetics of antigen-MHC molecule transfer from APC to T cells.

### EXAMPLES

The following examples further illustrate the invention but, of course, should not be construed as in any way limiting its scope.

### Example 1

This example describes the preparation of a human modified APC that stably expresses an MHC molecule-reporter peptide fusion protein that forms a complex with an antigen introduced into the APC. This example also describes the detectable transfer of such a complex from the APC to an antigen-specific T cell.

A full-length human leukocyte antigen (HLA) class I HLA-A*201) cDNA construct was obtained from RSV/HLA-A2 vector (Winter et al., J. Immunol. 146:3508-3512 (1991)). A HLA-A2-GFP expression vector was generated by inserting the HLA-A*201 cDNA with a stop codon mutated into the pEGFP-N3 vector (Clontech, Palo Alto, Calif.). A HLA-A- and HLA-B-locus-defective immortalized B cell line (Hmy2.CIR) was transfected with the HLA-A2-GFP vector using Trans-IT (Mirus, Madison, Wis.), according to the manufacture's instructions. The cells were incubated for 48 hours at 37° C., and subsequently replaced in selection medium (D-MEM supplemented with 10% fetal bovine serum, 2 mM 1-glutamine, 40 U/mL; penicillin, 40 μg/mL streptomycin (Gibco-BRL, Grand Island, N.Y.) and 400 μg/mL G418 Sulfate (Cellgro, Herndon, Va.)) to establish a stable cell line (HmyA2GFP) expressing an HLA-A*201-GFP fusion protein.

Previously described Tax11-19-specific CD8+ CTL clones (Kubota, et al., J. Immunol., 164:5192-5198 (2000)) were obtained and maintained by weekly stimulation with peptide-pulsed (1 μM) irradiated HLA-A*201 allogeneic PBMC at 1:10 CTL clone/PBMC ratio. Human rIL-2 (40 U/mL; Roche Diagnostic, Indianapolis, Ind.) was added to the cells on the day following stimulation. The CTLs were cultured in a “CTL medium” composed of Iscove's modified Dulbecco's medium (Gibco-BRL, Grand Island, N.Y.) supplemented with 10% human serum, 2 mM 1-glutamine, 40 U/ml penicillin and 40 μg/ml streptomycin.

HTLV-I Tax11-19 peptide (LLFGYPVYV) (SEQ ID NO: 1) and human immunodeficiency virus (HIV) Gag77-85 (SLYNTVATL) (SEQ ID NO:2) were synthesized and 95% purified by high-performance liquid chromatography (New England Peptide, Fitchburg, Mass.). HmyA2GFP cells were pulsed with HTLV-I Tax11-19 peptide or HIV GAG77-85 at a concentration of 10 μM for 30 minutes, and cultured with the above-described HTLV-I Tax11-19 specific CD8+ T cell clones on a poly-D-lysine-treated glass coverslip at 37° C. in a 5:1 cell ratio (HmyA2GFP cells/CTL=5/1). The cells were then fixed in 4% paraformaldehyde and stained with monoclonal antibody to CD8 (DAKO, Denmark) in combination with Alexa Fluor 350 goat anti-mouse IgG1 (blue) (Molecular Probes, Inc., Eugene, Oreg.). The cells were subjected to fluorescence activated cell sorter (FACS) analysis with a FACS Calibur instrument (Becton Dickinson, San Jose, Calif.) and/or examined with a Zeiss Axiovert 200M microscope.

The acquisition of peptide/HLA-GFP complexes by the HLA-A*201-restricted HTLV-I-specific CTL clones was readily observed by both fluorescence microscopy and FACS analysis. Such observation of the cells indicated that Hmy2.CIR cells were negative for expression of HLA-A*201 molecule and GFP, but HmyA2GFP cells expressed HLA-A*201 molecule and GFP in a colocalized manner. Within 5 minutes of mixing the cells, the HLA-GFP molecules formed dense clusters that could be readily visualized at the T cell-APC contact site. After 30 minutes, small aggregates of HLA-GFP appeared within the HTLV-I Tax11-19 peptide-specific CTL clones, at which point a majority of CD8+ cells that had acquired HLA-GFP were no longer in contact with the HmyA2GFP cells. FACS analysis also indicated that at about 30 minutes most HTLV-I-specific CTL clones mixed with Tax11-19-pulsed HmyA2GFP cells were positive for HLA-GFP, whereas Tax11-19-specific CTL clones mixed with Gag77-85-pulsed HmyA2GFP cells were negative for HLA-GFP.

To determine the kinetics of the transfer of the antigen-fusion protein complex from the APCs to the antigen-specific T cell clones, clones were incubated with HmyA2GFP cells pulsed with Tax11-19 peptide for various periods and the HLA-GFP acquisition by CTL was directly analyzed by flow cytometry. HIV Gag77-85 peptide-pulsed HmyA2GFP cells were similarly obtained and mixed with Tax11-19-specific T cell clones, and the T cells were subjected to similar FACS analysis. Data obtained from these experiments are set forth in Table 1 (the levels of HLA-GFP acquisition are expressed as mean fluorescence intensity (MFI)).

TABLE 1
Kinetics of GFP Complex Acquisition by Antigen-Specific T Cell Clones

| | MFI of clones mixed with | MFI of clones mixed with |
|---|---|---|
| | HmyA2GFP cells pulsed | HmyA2GFP cells pulsed |
| Time (hours) | with Tax11-19 | with Gag77-85 |
| 0 | 5.8 | 4.34 |
| 0.5 | 49.93 | 10.41 |
| 1 | 51.03 | 7.72 |
| 2 | 50.62 | 8.35 |
| 4 | 8.63 | 3.99 |
| 8 | 5.8 | 4.34 |

As shown in Table 1, after 30 minutes, the majority of the Tax11-19-specific CTL clones mixed with Tax11-19-pulsed HmyA2GFP cells were positive for HLA-GFP (as indicated by an at least about 5 fold increase in the number of cells). The level of GFP fluorescence in these antigen-specific T cell clones remained relatively constant for up to about 2 hours and thereafter declined. A dose-dependent titration of HTLV-I Tax11-19 peptide pulsed-HmyA2GFP cells demonstrated a lower limit of detection of 100 pM of peptide (data not shown). In contrast, control cells showed little change in MFI throughout the entire period.

This example describes a method for producing a modified human APC that expresses an HLA-A*201-reporter peptide fusion protein, which, upon introduction of an antigenic peptide, displays a complex of the fusion protein and peptide that is detectably internalized by antigen-specific T cells. This example also describes a method for determining the kinetics of MHC-antigen transfer from APCs to antigen-specific T cells.

### Example 2

This example demonstrates that antigen-specific T cells in a population of cells can be accurately quantified by observing the transfer of a detectable MHC molecule/reporter peptide fusion protein-antigen complex from modified APCs that display the complex to T cells specific for the antigen.

Human PBMCs were obtained by Fycoll-Hypaque centrifugation of blood samples obtained from HTLV-I-infected human patients afflicted with an inflammatory disease of the central nervous system termed HTLV-I-associated myelopathy/tropical spastic paraparesis (HAM/TSP). It has been previously demonstrated that a high frequency of HTLV-I-specific CD8+ CTL exists in HAM/TSP patients and that most of these CTLs recognize the HTLV-I Tax11-19 peptide. Jacobson et al., Nature 348:245-248 (1990), Bangham, Curr. Opin. Immunol. 12:397-402 (2000), and Yamano et al., Blood, 99:88-94 (2002). In addition, it has been suggested that these HTLV-I-specific CTL play an important role in the pathogenesis of this disease. PBMCs were similarly obtained from 3 HLA-A*201 negative HAM/TSP patients, and 3 HLA-A*201 positive HTLV-I seronegative healthy donors, to provide negative control PBMC populations. The diagnosis of HAM/TSP was assessed according to the WHO guidelines. Blood samples were obtained after informed consent as part of a clinical protocol reviewed and approved by the National Institutes of Health institutional review panel. Infection of HTLV-I was confirmed by enzyme linked immunosorbent assay (ELISA) (Abbot Labs, Chicago, Ill.) and Western blot analysis (Gene Labs, Singapore).

HmyA2GFP cells were pulsed with HTLV Tax11-19 peptide and incubated in a round bottom 96 well culture plate for 30 min at 37° C. The HmyA2GFP cells were thereafter washed twice to remove any free peptide, and separate populations of such HmyA2GFP cells were mixed in a round bottom 96 well culture plate with PBMCs obtained from one of the 8 HLA-A*201-positive HAM/TSP patients, the 3 HLA-A*201-negative HAM/TSP patients, and the 3 HLA-A*201-positive healthy donors in a 1:1 cell ratio (HmyA2GFP/PBMC=1/1). The HmyA2GFP/PBMC mixtures were centrifuged at 200 g for a few seconds to provide immediate contact of the cells, and thereafter incubated for 30 minutes at 37° C.

The PBMCs were stained with a saturating concentration of TC-labeled monoclonal antibody to CD8 (Caltag) at 4° C. for 30 minutes or PE-conjugated Tax11-19 peptide loaded HLA-A*201 tetramer (provided by National Institutes of Health AIDS Research and Reference Reagent Program). The cells were washed twice and subjected to FACS analysis with a FACS Calibur device. Data obtained from the FACS assays were analyzed with Cellquest software (Becton Dickinson).

Through the above-described FACS analysis, the percentage of CD8+GFP+ cells in each population of donor PBMCs mixed with HmyA2GFP cells pulsed with Tax11-19 peptide was determined. The frequency of Tax11-19-specific T cells mixed with HmyA2GFP cells pulsed with Tax11-19 peptide also was determined by tetramer analysis using HTLV-I Tax11-19/HLA-A*201 tetramers. The results of these studies are presented in Table 2.

TABLE 2
Percentage of HTLV-I Tax11-19-Specific T Cells As
Determined by % GFP Acquisition and Tetramer Analysis

| Patient | *% GFP acquisition | *% tetramer |
|---|---|---|
| A201(+) HAM #1 | 11.89 | 16.15 |
| A201(+) HAM #2 | 1.66 | 1.87 |
| A201(+) HAM #3 | 15.44 | 20.40 |
| A201(+) HAM #4 | 25.13 | 28.21 |
| A201(+) HAM #5 | 3.91 | 4.77 |
| A201(+) HAM #6 | 1.18 | 1.18 |
| A201(+) HAM #7 | 2.50 | 2.76 |
| A201(+) HAM #8 | 1.69 | 1.78 |
| A201(−) HAM #9 | 0.59 | 0.32 |
| A201(−) HAM #10 | 0.48 | 0.15 |
| A201(−) HAM #11 | 0.51 | 0.12 |
| A201(+) HD #1 | 0.43 | 0.12 |
| A201(+) HD #2 | 0.37 | 0.15 |
| A201(+) HD #3 | 0.33 | 0.13 |

HD: HTLV-I seronegative healthy donor
A201(+): HLA-A*201 positive individual
A201(−): HLA-A*201 negative individual

As illustrated in Table 1, the frequencies of HTLV-I Tax11-19-specific T cells detected in the PBMC populations by T cell acquisition, including the PBMCs obtained from the 8 HLA-A*201-positive HAM/TSP patients, were comparable with the frequency of HTLV-I Tax11-19-specific T cells in the PBMCs as determined by tetramer analysis. In over 50% of the PBMCs obtained HLA-A*201-positive HAM/TSP patients the frequency of antigen-specific T cells identified by the inventive method differed by less than 15% from the frequency determined by tetramer analysis and in 75% of such PBMCs the frequencies differed by less than 20%. The biggest difference between the frequency of antigen-specific T cells in the population differed by only about 25%. In the case of patient #6, no difference was observed between the frequencies of antigen-specific T cells. Consistent with the tetramer analysis, Tax11-19-specific T cells were not detected in HLA-A*201 negative patients nor in healthy donors by the inventive method.

The results of these experiments demonstrate that the frequency of antigen-specific T cells in a population of cells can be quantified by observing T cell acquisition of detectable fusion protein-antigen complexes from APCs displaying such complexes, in accordance with inventive methods described herein.

### Example 3

This example describes the quantification of the frequency of cytomegalovirus (CMV) antigen-specific T cells in a population of PBMCs by applying techniques in accordance with inventive methods described herein.

PBMCs were obtained from a blood sample taken from HLA-A*201 HAM/TSP patient #5 (see Example 2) by Fycoll-Hypaque centrifugation. HmyA2GFP cells were pulsed with 95% HPLC-purified synthetic CMV pp65 (495-503, NLVPMVATV) (SEQ ID NO:3) and thereafter mixed in a round bottom 96 well culture plate with the PBMCs in a 1:1 cell ratio (HmyA2GFP/PBMC=1/1), centrifuged at 200 g for a few seconds to provide immediate contact of the cells, and thereafter incubated for 30 min at 37<C. The PBMCs were stained with CMV pp65 peptide loaded HLA-A*201 tetramer (Beckman Coulter, Fullerton, Calif.) or phycoerythrin (PE)-labeled monoclonal antibody to CD8 (Caltag, Burlingame, Calif.). The cells were subjected to FACS analysis to identify the proportion of CMV pp65-specific T cells in the PBMCs by tetramer analysis and the inventive method.

FACS analysis of PBMCs labeled with CMV pp65 tetramer indicated that 6.57% of the cells in the PBMCs were CMV pp65-specific T cells. The results of cells subjected to the inventive method indicated that the frequency of CMV pp65-specific T cells in the PBMC population was 6.34%. Thus, the frequencies of CMV pp65-specific T cells determined by tetramer analysis and the method of the invention were also comparable with respect to a CMV antigen. Indeed, the differences in the frequency of CMV antigen-specific T cells obtained by the two techniques differed by less than 5%.

The results of this experiment further demonstrate that the number of antigen-specific T cells in a cell population can be determined in accordance with the present inventive method at levels and sensitivities comparable to detection by tetramer analysis.

### Example 4

This example demonstrates the identification of antigen-specific T cells and epitopes by applying methods in accordance with the present inventive method.

HLA-A*201 HAM/TSP (HAM #1) patient PBMCs (see Example 2) were obtained and incubated with HmyA2GFP cells pulsed with a series of overlapping peptides from the HTLV-I Env gp46 region (see Frangione-Beebe, Vaccine 19:1068-1081 (2000)). HLA-GFP acquisition by CD8+ T cells was analyzed by flow cytometry as described in Example 2 to determine the frequency of antigen-specific T cells in the population of PBMCs.

The results of these experiments are set forth in Table 3 (each HTLV-I Env peptide is designated by the position of its amino acid sequence).

TABLE 3
Identpication of Antigenic Portions of a Viral Polypeptide by Determining
the Percentage of T cells Acquiring HLA-GFP Fusion Protein upon
Stimulation with an APC Displaying a Complex of the Peptide and
the HLA-GFP Fusion Protein

| | % HLA-GFP | | % HLA-GFP |
|---|---|---|---|
| | acquisition in | | acquisition in |
| Peptide | CD8+ Cells | Peptide | CD+ Cells |
| Env(11-25) | 0.5 | Env(151-165) | 3.87 |
| Env(21-35) | 0.6 | Env(161-175) | 3.52 |
| Env(31-45) | 0.5 | Env(171-185) | 3.37 |
| Env(51-65) | 0.75 | Env(191-205) | 2.25 |
| Env(61-75) | 1.49 | Env(201-215) | 2.33 |
| Env(71-85) | 3.27 | Env(231-245) | 2.9 |
| Env(81-95) | 1.41 | Env(241-255) | 2.6 |
| Env(95-105) | 1.15 | Env(251-265) | 3.63 |
| Env(101-115) | 1.44 | Env(261-275) | 3.01 |
| Env(111-125) | 1.91 | Env(281-295) | 3.43 |
| Env(121-135) | 1.61 | Env(291-305) | 9.07 |
| Env(131-145) | 2.94 | Env(301-315) | 3.23 |
| Env(141-155) | 3.16 | Tax(11-19) | 13.95 |

As shown in Table 3, significant HLA-GFP acquisition was observed for CD8+ T cells mixed with HmyA2GFP cells pulsed with the Env(291-305) peptide. These data indicate that the Env(291-305) peptide is or comprises an epitope for T cells in the tested PBMCs.

To confirm this observation, a mixture Env(291-305) peptide-pulsed HmyA2GFP cells and PBMCs was stained with anti-CD8 monoclonal antibody and HLA-GFP acquiring CD8+ T cells were sorted therefrom by flow cytometry using a FACS Vantage SE sorter (Becton Dickinson). The sorted CD8+ T cells were incubated in CTL culture medium (see Example 1) overnight. The cultured cells were subjected to a 4-hour CTL assay performed with Europium (Aldrich Chemical, Milwaukee, Wis.) as described previously (Kubota et al., J. Immunol. 164:5192-5198 (2000)). T cells were incubated with target cells at an effector-to-target cell ratio of 3:1. Target cells consisted of HLA-A*201 transfected human B cell line pulsed with Env(11-25), Env(291-305), Tax11-19 peptide and CMV pp65 peptide at a peptide concentration of 100 nM, HLA-A*201 transfected human B cell line infected with vaccinia recombinant viruses expressing HTLV-I Env or a control vaccinia expressing influenza virus hemagglutinin (HA) protein, and an autologous HTLV-I infected CD4+ T cell line known to express HTLV-I (RSCD4) (Mendez et al., J. Virol. 71, 9143-9149 (1997)). The % specific lysis was calculated as (experimental release−spontaneous release)/(maximum release−spontaneous release)×100. The assay was performed in triplicate. The results of these experiments are presented in Table 4.

TABLE 4
Specific Lysis of Target Cells by Antigen-Specific T Cells

| Target Cell | % Specific Lysis |
|---|---|
| Env(11-25) | 2.5 |
| Env(291-305) | 16.7 |
| Vaccinia HA | 3.7 |
| Vaccinia Env | 22.6 |
| RS CD4 | 32.4 |

HTLV-I Env291-305-pulsed target cells were significantly lysed compared to target cells pulsed with the Env(11-25) control peptide (as evidenced by at least about 6 times more specific lysis of target cells). Moreover, target cells infected with a Vaccinia-Env or an autologous HTLV-I infected CD4+ T cell line (RS CD4) known to express HTLV-I (ref. 11) were also lysed by the Env291-305 sorted CD8+ T cells.

These results clearly showed that the sorted Env291-305-specific CD8+ T cells detected by the A2GFP system were peptide-specific and functionally cytolytic, and indicate that the Env291-305 region of HTLV-I contains an immunodominant, HLA-A*201-specific CTL epitope. Of interest is the observation that this Env291-305 region contained a relatively strong HLA-A*201 binding motif (Parker et al., J. Immunol. 152, 163-175 (1994)) based on an estimation of the dissociation rate of the peptide-HLA complex obtained using the publicly available BIMAS HLA Peptide Binding Predictions Program.

This example demonstrates that APCs expressing MHC molecule/reporter peptide fusion proteins can be used to identify T cell antigens, including CD8+ immunodominant epitopes, in a pool, panel, or population of peptides. This example further demonstrates that particular methods of the invention can be used to obtain a population of T cells specific for an antigen in a pool of polypeptides from a larger population of cells, such as a pool of PBMCs.

### Example 5

This example demonstrates the phenotypic and functional characterization of HTLV-I Tax11-19-specific and CMV pp65-specific T cells in PBMCs obtained from a HAM/TSP patient and the observation of changes in such phenotypic and functional characteristics in response to antigen stimulation.

The distribution of T cell differentiation markers CD27 and CD45RA on HTLV-I Tax11-19 tetramer-positive T cells and CMV pp65 tetramer-positive T cells were analyzed using PBMC from HAM/TSP patients (specifically, HTLV-I Tax11-19 tetramer positive cells were obtained from PBMC of HAM/TSP patients #5 and #7—see Example 2—and CMV pp65 tetramer-positive cells were obtained from PBMC of 4 HAM/TSP patients). CD27 and CD45RA molecules have been reported to distinguish phenotypic subpopulations of CD8+ T cells. Hamann et al., J. Exp. Med. 186:1407-1418 (1997). On the basis of both phenotypic and functional maturation properties, 4 distinct T-cell subsets have been described: CD27+CD45RA+ (naïve), CD27+CD45RA− (memory), CD27-CD45RA− (effector/memory), and CD27-CD45RA+ (effector) (Id.). The Tax11-19 and CMV pp65 tetramer-positive cells were labeled with PE-conjugated Tax11-19 peptide loaded HLA-A*201 tetramer or CMV pp65 loaded HLA-A*201 tetramer, respectively, monoclonal antibody to CD27, and allophycocyanin-labeled monoclonal antibody to CD45RA. Peridinine chlorophyll protein (PerCP)-labeled Rat Anti-Mouse IgG1 antibody was used as a secondary reagent for anti-CD27 antibody (all from BD Pharmingen, San Diego, Calif.). The cells were then analyzed by flow cytometry to determine their phenotypic profile with respect to these cell markers.

To study how virus-specific T cells with different phenotypes respond to antigen stimulation, alterations of CD27 and CD45RA expression in HLA-GFP-acquiring HTLV-I- and CMV tetramer-positive T cells after ex vivo peptide presentation by HmyA2GFP cells also was determined by FACS analysis. PBMCs obtained from HAMTSP patients were incubated with HTLV-I Tax11-19 peptide-pulsed or CMV pp65 peptide-pulsed HmyA2GFP cells for 1 hour and similarly subjected to flow cytometry analysis for these cell markers. The results of these experiments are presented in Tables 5 and 6 below.

TABLE 5
Cell Surface Marker Phenotypic Analysis
of HTLV-I tetramer-positive cells

| | % CD27+ | % CD27+ | % CD27− | % CD27− |
|---|---|---|---|---|
| | CD45RA+ | CD45RA− | CD45RA− | CD45RA+ |
| Before | 27.907 +/− | 50.063 +/− | 12.357 +/− | 9.671 +/− |
| contact with | 5.928* | 5.25 1 | 2.39 | 2.706 |
| HmyA2GFP | | | | |
| cells | | | | |
| After | 10.074 +/− | 5.8 +/− | 30.696 +/− | 53.429 +/− |
| contact with | 0.915 | 1.966 | 7.169 | 8.024 |
| HmyA2GFP | | | | |
| cells | | | | |

*standard error of the mean (s.e.m.)

TABLE 6
Cell Surface Marker Phenotypic Analysis
of CMV tetrarner-positive cells

| | % CD27+ | % CD27+ | % CD27− | % CD27− |
|---|---|---|---|---|
| | CD45RA+ | CD4SRA− | CD45RA− | CD45RA+ |
| Before | 18.707 +/− | 28.325 +/− | 8.44 +/− | 44.53 +/− |
| contact with | 8.173 | 17.986 | 2.704 | 13.153 |
| HmyA2GFP | | | | |
| cells | | | | |
| After | 3.05 +/− | 2.51 +/− | 51.813 +/− | 42.625 +/− |
| contact with | 0.713 | 0.816 | 12.08 | 12.37 |
| HmyA2GFP | | | | |
| cells | | | | |

As illustrated in Table 5, after stimulation with Tax11-19-pulsed HmyA2GFP cells, a decrease in the CD27+CD45RA− (memory) subset and an increase in the CD27-CD45+ (effector) subset was observed in HLA-GFP-acquiring HTLV-I tetramer-positive cells. In contrast, in HLA-GFP acquiring CMV tetramer-positive T cells, there was no significant change in the proportion of effector T cells after the stimulation with CMV pp65 peptide-pulsed HmyA2GFP cells (Table 6). However, there was a significant increase in CD27-CD45RA− (effector/memory) subset with a concomitant decrease in the CD27+CD45RA− (memory) subset (Table 6). Thus, although virus-specific populations during persistent HTLV-I infection were represented in each of the four different phenotypic subsets, HTLV-I Tax11-19 tetramer positive T cells demonstrated a clear enrichment of the CD27+CD45RA− (memory) subset. By contrast, CMV tetramer-positive T cells showed a substantial enrichment of the CD27-CD45RA+ (effector) subset.

The above-described phenotypically defined T cell populations were further characterized with respect to perforin expression and cytolytic activity. For intracellular perforin staining, cells were fixed and permeabilization with Cytofix/CytoPerm kit (BD Pharmingen), and stained with monoclonal antibody specific for perforin (BD Pharmingen). The cells were subjected to further FACS analysis and the resulting data was analyzed with Cellquest software (Becton Dickinson) to obtain the mean fluorescence intensity (MFI) for the cell populations. The results of these experiments are set forth in Table 7.

TABLE 7
Perforin Expression Levels in Phenotypically
Characterized T Cells (in MFI)

| | HLA acquiring | | HLA acquiring |
|---|---|---|---|
| HTLV-I tetramer- | HTLV-I tetramer | CMV tetramer- | CMV tetramer- |
| positive cells | positive cells | positive cells | positive cells |
| 11.56 +/− | 10.45 +/− | 43.832 +/− | 9.543 +/− |
| 2.175* | 0.805 | 8.177 | 1.746 |

*s.e.m.

The results of the experiments presented in Table 7 are consistent with previous reports indicating that terminally differentiated effector cells contain more perforin than cells with a naive or memory phenotype. See, e.g., Appay et al., supra. These results indicate that CMV-specific CD8+ T cells had higher levels of perforin than HTLV-I-specific CD8+ T cells ex vivo. In contrast, there was no change in the amount of perforin in HTLV-I-specific T cells after Tax11-19 stimulation, and there was a significant decrease in the amount of perforin in CMV-specific T cells after CMVpp65 peptide stimulation.

The phenotypically defined T-cell populations were further characterized with respect to cytolytic activity. HTLV-I tetramer-positive and CMV pp65 tetramer-positive T cells were sorted by flow cytometry and examined for the functional capacity to lyse peptide-pulsed target cells in the above-described 4 hour CTL assay (using effector-to-target cell ratio of 3:1—see Example 4). HTLV-I tetramer-positive cells exhibited about 12% specific lysis (11.7%), whereas CMV tetramer positive cells exhibited about 20% specific lysis (21.2%). Thus, as would be predicted based on the above-described functional characterization, CMV tetramer-positive T cells induced significantly higher cell lysis (e.g., about twice as much cell lysis) than HTLV-I tetramer-positive T cells.

PBMCs obtained from HAM/TSP patient #5 also were examined for their ability to lyse Tax11-19-pulsed target cells or CMV pp65-pulsed target cells in the above-described 4 hour CTL assay at various effector-to-target cell ratios. Specific lysis of non-peptide-pulsed target cells by PBMCs was used as a negative control. The results of these experiments are presented in Table 8.

TABLE 8
Specific Lysis of Target Cells by PBMCs
Obtained From a HAM/TSP Patient

| | E/T cell ratio | | | |
|---|---|---|---|---|
| Peptide | 100: 1 | 30: 1 | 10: 1 | 3: 1 |
| CMV pp65 | 26.2 | 11.4 | 5.5 | 2.6 |
| Tax11-19 | 11.2 | 3.5 | 1.8 | 2.4 |
| No peptide | 2.9 | 1.8 | 1.9 | 2.3 |

Consistent with the results of the above-described assays, bulk PBMC from a HAM/TSP patient exhibited substantially more cell lysis of target cells than HTLV-I tetramer positive cells.

The results of these experiments illustrate that inventive methods described herein can be used to define the differences in phenotype and functional characteristics of antigen-specific T cells in association with antigen stimulation. After antigen stimulation with peptide-pulsed HmyA2GFP cells, HTLV-I-specific CD8+ cells were enriched for an effector phenotype without a significant induction of perforin or increased cytolytic activity, suggesting that antigen stimulation of HTLV-I-specific CD8+ tell cells may be insufficient to control viral infection, resulting in increased viral load in HAM/TSP patients (see Champagne et al., supra (see also Yamano et al., Blood 99, 88-94 (2002) for discussion of related topics). In contrast, CMV-specific T cells exhibited phenotypic and functional characteristics consistent with a highly effective antivirus CD8+ cell-mediated immune response. Thus, these experiments also demonstrate that the acquisition of peptide/HLA A2-GFP by T cells specific for HTLV-I or CMV antigens can define stages of T cell differentiation between two viruses associated with chronic viral infections. Similar assays may be used as a tool to assess therapies based upon administration, stimulation, and/or promotion of antigen-specific T cells.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The phrase “consisting essentially of” where used herein, is intended to only limit the scope of the invention to the specified materials or steps and those that do not materially affect the basic and novel characteristics of the claimed invention as set forth above. As such, the scope of the invention where “consisting essentially of” is recited is intended to be narrower than where “comprising” is used, however broader than where “consisting of” is used. One of skill in the art, in reviewing the present specification, can readily identify those materials and steps that do not materially affect the basic and novel characteristics of the present invention.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An antigen presenting cell (APC) expressing at least two fusion proteins, wherein (i) each of the fusion proteins comprise an MHC molecule portion and a reporter peptide portion, and (ii) the reporter peptide portion of each fusion protein is detectably different from the reporter peptide portion of every other of the at least two fusion proteins and the MHC molecule portion of each fusion protein is different from the MHC molecule portion of every other of the at least two fusion proteins.

2. The APC of claim 1, wherein the reporter peptide portions of the at least two fusion proteins are fluorescent and fluoresce at detectably different wavelengths from one another.

3. The APC of claim 1, wherein the APC is prepared from a human cell.

4. A human APC that expresses a fusion protein comprising a human leukocyte antigen HLA-A*201 portion and a reporter peptide portion.

5. The APC of claim 4, wherein the reporter peptide portion comprises the amino acid sequence of a green fluorescent protein.

6. A method for determining whether a T cell specific for a peptide is present in a population of cells, which method comprises:
(a) introducing the peptide into an APC, which expresses a fusion protein comprising (i) a major histocompatibility complex (MHC) molecule portion and (ii) a reporter peptide portion, the peptide such that a complex forms between the fusion protein and the peptide and the peptide is displayed by the APC,
(b) contacting the APC displaying the complex with a population of cells, such that T cells in the population of cells specific for the antigen will detectably internalize the complex,
(c) determining T cells in the population of cells have detectably internalized the complex,
whereupon T cells which are in the population of cells and are specific for the peptide are determined to be present.

7. The method of claim 6, wherein the population of cells are human cells.

8. The method of claim 6, wherein the population of cells consists essentially of human peripheral blood mononuclear cells (PBMCs).

9. The method of claim 6, wherein the method comprises repeating steps (a)-(c) until T cells in the population have internalize the complex, whereupon a T cell specific for the antigen is identified, wherein a different population of cells contacts the APC displaying the complex in each repetition of steps (a)-(c).

10. A method for quantifying the number of T cells, which are specific for an epitope of interest, in a population of cells comprising T cells, which method comprises:
(a) introducing a peptide comprising the epitope of interest into an APC, which expresses a fusion protein comprising (i) an MHC molecule portion which binds an epitope of interest and (ii) a reporter peptide portion, a peptide comprising the epitope of interest such that a complex forms between the fusion protein and the peptide and the peptide is displayed by the APC,
(b) contacting the APC displaying the peptide with a population of cells comprising T cells, such that those T cells in the population of cells specific for the epitope detectably internalize the complex, and
(c) determining how many T cells in the population of cells have internalized the complex, whereupon T cells, which are in the population of cells and are specific for the epitope of interest, are quantified.

11. The method of claim 10, wherein the population of cells consists essentially of human PBMCs.

12. The method of claim 10, wherein the reporter peptide portion comprises the amino acid sequence of a fluorescent polypeptide.

13. The method of claim 10, wherein the method comprises comparing the number of T cells determined in step (d) with an enumeration of the antigen-specific T cells in the population obtained through tetramer analysis or cytokine secretion analysis.

14. The method of claim 10, wherein the T cells are primate T cells.

15. The method of claim 14, wherein the T cells are human T cells.

16. The method of claim 12, wherein the peptide is introduced into the APC by (a) expressing a nucleic acid in the APC that encodes a polypeptide that comprises the peptide or (b) contacting the APC with a polypeptide that comprises the peptide, wherein the APC processes the polypeptide to produce the peptide.

17. A method for determining whether a peptide induces a T cell-mediated immune response comprising:
(a) introducing the peptide into an APC, which expresses a fusion protein comprising (i) an MHC molecule portion and (ii) a reporter peptide portion, the peptide under conditions where a complex can form between the fusion protein and the peptide and the peptide is displayed by the APC,
(b) contacting the APC displaying the peptide with a population of T cells, such that T cells in the population specific for the peptide detectably internalize the complex, and
(c) detecting whether cells in the population of T cells have internalized the complex,
whereupon the peptide is determined to induce a T cell-mediated immune response.

18. The method of claim 17, wherein the peptide is a portion of a polypeptide.

19. The method of claim 18, wherein the polypeptide is known to induce a T cell response.

20. The method of claim 17, wherein the peptide is introduced into the APC by (a) expressing a nucleic acid in the APC that encodes a polypeptide that consists essentially of the peptide or (b) contacting the APC with a polypeptide that consists essentially of the peptide, wherein the APC processes the polypeptide to produce the peptide.

21. The method of claim 20, wherein the nucleic acid comprises part of a viral genome.

22. The method of claim 21, wherein the viral genome is a recombinant viral genome and the polypeptide is a nonviral polypeptide or a viral polypeptide not expressed by the viral genome associated with the virus from which the genome was obtained or derived.

23. The method of claim 18, wherein the polypeptide is selected from a number of polypeptides collectively known to induce a T cell response.

24. The method of claim 17, wherein the peptide introduced into the APC is about 7-25 amino acids in length.

25. The method of claim 17, wherein the method comprises repeating steps (a)-(c) each time with a different peptide, until T cells in the population of T cells have internalized the complex.

26. The method of claim 25, wherein a portion of the amino acid sequence of each peptide is identical to a portion of the amino acid sequence of at least one other peptide.

27. The method of claim 17, wherein the peptide is associated with an autoimmune disease in a human, a cancer or a virus.

28. The method of claim 17, wherein the peptide is an altered peptide ligand (APL) derived from an antigenic peptide by the addition, substitution, or deletion of one or more amino acid residues in the antigenic peptide.

29. A method of monitoring the efficacy of treatment of a disease in a patient, which method comprises comparing the number of T cells, which are specific for one or more epitopes of interest, which can be from one or more antigens of interest, in a population of cells comprising T cells obtained from the patient before treatment and in a population of cells comprising T cells obtained from the patient during and/or after treatment,
wherein the number of T cells are determined in accordance with the method of claim 10,
wherein, when the treatment induces a T cell-mediated response, an increase in the number of T cells after treatment as compared to the number of T cells before treatment indicates that the treatment is efficacious, whereas no change in the number of T cells or a decrease in the number of T cells after treatment as compared to the number of T cells before treatment indicates that the treatment is not efficacious, and
wherein, when the treatment inhibits a T cell-mediated response, no change in the number of T cells or a decrease in the number of T cells after treatment as compared to the number of T cells before treatment indicates that the treatment is efficacious, whereas an increase in the number of T cells after treatment as compared to the number of T cells before treatment indicates that the treatment is not efficacious.

30. The method of claim 29, wherein the disease is mediated by a pathological T cell response.

31. The method of claim 29, wherein:
(a) the disease is an autoimmune disease and the one or more epitopes of interest is/are autoimmune epitope(s);
(b) the disease is diabetes and the one or more epitopes of interest is/are epitopes from an islet of Langerhans cell;
(c) the disease is arthritis and the one or more epitopes of interest is/are from collagen;
(d) the disease is multiple sclerosis and the one or more epitopes of interest is/are from myelin or an antigen of the central nervous system;
(e) the disease results from infection with a virus and the one or more epitopes of interest is/are from the virus; or
(f) the virus is human immunodeficiency virus (HIV), a species of Vaccinia, hepatitis virus, or cytomegalovirus (CMV).

32. The method of claim 31, wherein the disease results from infection with a bacterium and the one or more epitopes of interest is/are from the bacterium.

33. The method of claim 32, wherein the bacterium is a species of Chlamydia, Helicobacter or Mycobacteria.

34. The method of claim 29, wherein the disease results from infection with a parasite and the one or more epitopes of interest is/are from the parasite.

35. The method of claim 29, wherein the treatment comprises vaccination or immunization against the disease.

36. The method of claim 29, wherein the disease is anthrax, measles, rubella or cancer.

37. A method of evaluating the immunological effect of an antigen on the phenotypic or functional activity profile of a population of T cells comprising:
(a) introducing the antigen into an APC, which expresses a fusion protein comprising (i) an MHC molecule portion and (ii) a reporter peptide portion, the antigen such that a complex forms between the fusion protein and the antigen and the antigen is displayed by the APC,
(b) contacting the APC displaying the antigen with a population of T cells comprising T cells specific for the antigen and characterized by a phenotypic trait or functional trait, such that at least some of the T cells detectably internalize the complex, and
(c) characterizing the population of T cells on the basis of (i) the phenotypic trait, (ii) the functional trait, (iii) a second phenotypic trait that differs from the phenotypic trait, (iv) a second functional trait that differs from the functional trait, or (v) any combination of (i)-(iv),
whereupon the immunological effect of the antigen on the phenotypic or functional activity profile of the population of T cells is evaluated.

38. The method of claim 37, wherein the method comprises characterizing the population of T cells on the basis of the number of T cells in the population that comprise one or more cell surface markers.

39. The method of claim 38, wherein the one or more cell surface markers are selected from the group consisting of CD27, CD28, CCR7, CD45RA, CD45RO, and combinations thereof.

40. The method of claim 37, wherein the method comprises characterizing the population of T cells on the basis of perforin expression level.

41. The method of claim 37, wherein the T cells are cytolytic lymphocytes (CTLs) and the method comprises characterizing the population of T cells on the basis of the cytolytic activity of the T cells with respect to one or more target cells.

42. The method of claim 37, wherein the T cells are CD4+ T cells and the method comprises characterizing the T cells on the basis of cytokine and/or chemokine secretion from or expression in the T cells.

43. The method of claim 37, wherein the phenotypic trait or functional trait detectably changes in association with the maturity of the T cells, whereupon the method provides a method for assessing the maturity level of the T cells.

44. The method of claim 37, wherein the antigen is associated with disease-causing cells and the method further comprises repeating step (c) over time and characterizing the population of T cells on the basis of (i) the phenotypic trait or (ii) the functional trait, whereupon the effectiveness of a T-cell-mediated response to the antigen is assessed.

45. The method of claim 37, wherein the phenotypic trait or functional trait detectably changes as T cells mature and the method further comprises repeating step (c) over time and characterizing the population of T cells on the basis of (i) the phenotypic trait or (ii) the functional trait, whereupon the maturity of the T cells is assessed.

46. A method of preparing a targeted pharmaceutical composition for ameliorating a disease associated with T cell activity in a mammal comprising performing the method of claim 20, characterizing the peptide that induces a T cell-mediated immune response and associating (i) an antigenic peptide comprising an amino acid sequence consisting essentially of the amino acid sequence of the amino acid sequence of the peptide and (ii) a molecule that inhibits the proliferation and/or activity of T cells, whereupon a targeted pharmaceutical composition for ameliorating a disease associated with T cell activity in a mammal is prepared.

47. The method of claim 46, wherein the antigenic peptide comprises the amino acid sequence of the peptide internalized by the cells.

48. The method of claim 46, wherein the antigenic peptide is conjugated to a toxin.

49. The method of claim 46, wherein the antigenic peptide comprises a first peptide portion comprising the amino acid sequence of the peptide internalized by the T cells fused to a second peptide portion comprising an amino acid sequence that promotes apoptosis of T cells.

50. The method of claim 46, wherein the disease is an autoimmune disease that afflicts humans.
